(19) United States
(12) Patent Application Publication
(10) Pub. No.: US 2022/0175571 A1
(43) Pub. Date: Jun. 9, 2022

(54) Prefabricated Customizable Cranial Remodelling Orthotic

(71) Applicant: Jason Shane Goodnough, New Westminster (CA)

(72) Inventor: Jason Shane Goodnough, New Westminster (CA)

(21) Appl. No.: 17/535,627

(22) Filed: Nov. 25, 2021

Related U.S. Application Data
(60) Provisional application No. 63/123,052, filed on Dec. 9, 2020.

(51) Int. Cl. A61F 5/058

(52) U.S. Cl. CPC A61F 5/05883; A61F 2005/0172

(57) ABSTRACT

A cranial remodeling orthotic for passive remodeling of a cranium of an infant has (i) a main frame assembly for encircling the infant cranium (ii) at least one support member having a concave inner surface to extend circumferentially with the main frame assembly about a portion of the infant cranium while protruding beyond at least one of the upper boundary or the lower boundary of the main frame assembly, and (iii) a resilient cushion layer lining an inner surface of a portion of the main frame assembly or the support member. The support member is rigid and self-supporting, while being formable in shape upon the application of heat more readily than the main frame assembly. The resulting orthotic can be mass produced while still allowing some customization for the end user so as to effectively treat headshape asymmetry without the need for 3D scans or plaster casting.

This application claims the benefit under 35 U.S.C.119(e) of U.S. provisional application Ser. No. 63/123,052, filed Dec. 9, 2020.

### FIELD OF THE INVENTION

The present invention relates to a cranial remodelling orthotic that can be prefabricated to generic specifications and customized to an individual patient after fabrication.

### BACKGROUND

Due to the infant craniums remaining soft and malleable at birth, the infant can develop flat spots on the cranium over time as a result of the infant being placed in the same positions such as on their back for extended periods of time. Plagiocephaly is a condition characterized by an asymmetrical distortion of the cranium resulting in a flat spot offset to one side at the back of the cranium. Brachycephaly is a condition characterized by a symmetrical flattening at the back of the cranium. Scaphocephaly is a condition where there is flattening on the right and left sides of the cranium.

Plagiocephaly, Brachycephaly and Scaphocephaly can each be treated by Orthotic Cranioplasty Treatment that typically involves custom manufacturing of an orthotic that is specially fitted to the patient. Such custom orthotic are costly and require specialists to measure and fit the orthotic to the patient. Accordingly, millions of infants around the world do not have access to Orthotic Cranioplasty Treatment and will have lifelong complications of pediatric head shape asymmetry.

### SUMMARY OF THE INVENTION

According to one aspect of the invention there is provided a cranial remodeling orthotic for passive remodeling of a cranium of an infant, the orthotic comprising:
a main frame assembly forming an annular band having an upper boundary and a lower boundary so as to be arranged to encircle the cranium of the infant;
at least one support member supported on the main frame assembly and having a concave inner surface, said at least one support member being arranged to extend circumferentially with the main frame assembly about a respective portion of the cranium of the infant, and said at least one support member protruding beyond at least one of the upper boundary or the lower boundary of the main frame assembly;
said at least one support member being formed of a support material that is rigid and self supporting; and
a cushion layer formed of resilient material lining an inner surface of at least a portion of the main frame assembly or said at least one support member;
the support material of said at least one support member being formable in shape upon the application of heat; and
said at least one support member being sized and configured so as to be formable in shape upon the application of heat more readily than the main frame assembly.

Preferably said at least one support member protrudes above the upper boundary of the main frame assembly, for example about a full circumference of the main frame assembly.

Preferably said at least one support member protrudes below the lower boundary of the main frame assembly, for example about a full circumference of the main frame assembly.

Preferably said at least one support member is perforated by an array of openings.

When the main frame assembly and said at least one support member are both formed of said support material, preferably the main frame assembly has fewer openings therein than said at least one support member such that said at least one support member is formable in shape upon the application of heat more readily than the main frame assembly.

Preferably said at least one support member has at least one of an upper free edge above the main frame assembly or a lower free edge below the main frame assembly that can be curled outward relative to the main frame assembly upon the application of heat.

Preferably said at least one support member fully spans an inner side of the main frame assembly.

The support member(s) may be formed integrally and seamlessly of the same material with a respective portion of the main frame assembly.

Alternatively, the support member(s) and the main frame assembly may be formed separately and laminated together during manufacturing.

The main frame assembly preferably comprises a plurality of arcuate sections connected circumferentially in series with one another to form the annular band in which said at least one support member comprises one or more support members supported on each arcuate section of the main frame.

More particularly, the main frame assembly may comprise (i) a plurality of arcuate sections connected circumferentially in series with one another at a plurality of junctions to form the annular band and (ii) an adjustable connector at each junction between a respective adjacent pair of the arcuate sections, in which each adjustable connector is adjustable in length in a circumferential direction of the annular band and is securable to join the adjacent pair of arcuate sections in fixed relation to one another, Preferably the arcuate sections and the adjustable connectors enable the annular band to be adjusted in length between a front and a rear of the annular band and to be adjusted in width transversely to the length.

Preferably each adjustable connector is operable between an open position limiting relative movement between the arcuate sections of the respective adjacent pair to linear sliding movement in the circumferential direction and a closed position securing the arcuate sections of the respective adjacent pair in fixed relation to one another.

When the main frame assembly comprises a plurality of arcuate sections connected circumferentially in series with one another to form the annular band, the plurality of arcuate sections preferably include (i) a rear section occupying a rear of the annular band for alignment with a rear of the cranium of the infant and (ii) two side sections extending forwardly from opposing ends of the rear section respectively so as to occupy opposing side portions of the annular band respectively, in which the two side sections are joined to one another at a front of the annular band for alignment with a forehead of the cranium of the infant.

According to a second aspect of the present invention there is provided a cranial remodeling orthotic for passive remodeling of a cranium of an infant, the orthotic comprising:
a main frame assembly forming an annular band arranged to encircle the cranium of the infant;
at least one support member supported on the main frame assembly and having a concave inner surface, said at least one support member being arranged to extend circumferentially with the main frame assembly about a respective portion of the cranium of the infant, said at least one support member being formed of a support material that is rigid and self supporting; and
a cushion layer formed of resilient material lining an inner surface of at least a portion of the main frame assembly or said at least one support member;
the main frame assembly comprises a plurality of arcuate sections connected circumferentially in series with one another to form the annular band, the plurality of arcuate sections including:
   (i) a rear section occupying a rear of the annular band for alignment with a rear of the cranium of the infant; and
   (ii) two side sections extending forwardly from opposing ends of the rear section respectively so as to occupy opposing side portions of the annular band respectively;
the two side sections being joined to one another at a front of the annular band for alignment with a forehead of the cranium of the infant.

The cushion layer according to any aspect of the invention may comprise a cushion pad supported on each of the two side sections and at least one control pad releasably supported on the rear section.

The at least one control pad may comprise a plagiocephaly control pad arranged to be supported along only one side of the rear section of the annular band, in which the plagiocephaly control pad is releasably supported on the rear section so as to allow placement on the rear section adjacent to either one of the two side sections. Preferably the cushion pads and the plagiocephaly control pad are formed of a common foam material and have a common thickness in a radial direction of the annular band, and the plagiocephaly control pad is circumferentially abutted against one of the cushion pads.

The at least one control pad may comprise a scaphocephaly control pad arranged to be supported directly in the mid-line of the rear section of the annular band, in which the scaphocephaly control pad is releasably supported on the rear section so as to allow placement on the rear section adjacent to either one of the two side sections. Preferably the cushion pads and the scaphocephaly control pad are formed of a common foam material and have a common thickness in a radial direction of the annular band, and the scaphocephaly control pad is placed evenly spaced between the two side cushion pads.

Alternatively, the at least one control pad may comprise two brachycephaly control pads arranged to be supported at circumferentially spaced positions on the rear section of the annular band, adjacent to the two side sections of the annular band respectively. Preferably the cushion pads and two brachycephaly control pads are formed of a common foam material and have a common thickness in a radial direction of the annular band, two brachycephaly control pads being circumferentially abutted against the two cushion pads respectively.

The orthotic described herein allows manufacturing of a mass market Cranial Orthosis and an appropriate remote support framework to those without access to traditional custom Orthotic Cranioplasty Treatment. The orthotic effectively treats headshape asymmetry without the need for individual infant head-modelling inputs such as 3D scans or plaster casting.

The Orthosis will use traditionally proven biomechanical principles to improve infant head symmetry by protecting from further external force deformation and directing future growth to skull areas lacking volume. The Orthosis will be designed with infant-appropriate aesthetic and safety principles, prefabricated for efficient delivery, have a defined life span, for example a 10 weeks duration, and will be adjustable using common household items to ensure comfort and growth accommodation can be achieved remotely by the parents or local healthcare provider.

The target population is infants with headshape asymmetry between 4 and 12 months of age at initiation of treatment with average head size.

### BRIEF DESCRIPTION OF THE DRAWINGS

One embodiment of the invention will now be described in conjunction with the accompanying drawings in which:

FIG. 1 is a perspective view of the cranial remodelling orthotic according to the present invention, in which some of the pads of the cushioning layer have been removed for illustrative purposes;

FIG. 2 is a left side elevational view of the orthotic according to FIG. 1 in which the opposing right side view is substantially identical to the left side view that is shown;

FIG. 3 is a rear view of the orthotic according to FIG. 1;

FIG. 4 is a top view of the orthotic according to FIG. 1;

FIG. 5 is a bottom view of the orthotic according to FIG. 1;

FIG. 6 is a perspective view of a plagiocephaly control pad which is positioned within the orthotic according to the broken line outline in FIG. 1;

FIG. 7 is a sectional view along a horizontal plane showing a first placement of the plagiocephaly control pad in solid line and a second alternatively placement of the plagiocephaly control pad in broken line;

FIG. 8 is a perspective view of two brachycephaly control pads which can be positioned in the orthotic according to FIG. 1 in place of the plagiocephaly control pad;

FIG. 9 is a sectional view along a horizontal plane showing placement of the brachycephaly control pads in the orthotic according to FIG. 1; and

FIG. 10 is a sectional view along a vertical plane through one of the adjustable connectors of the main frame assembly within the orthotic according to FIG. 1.

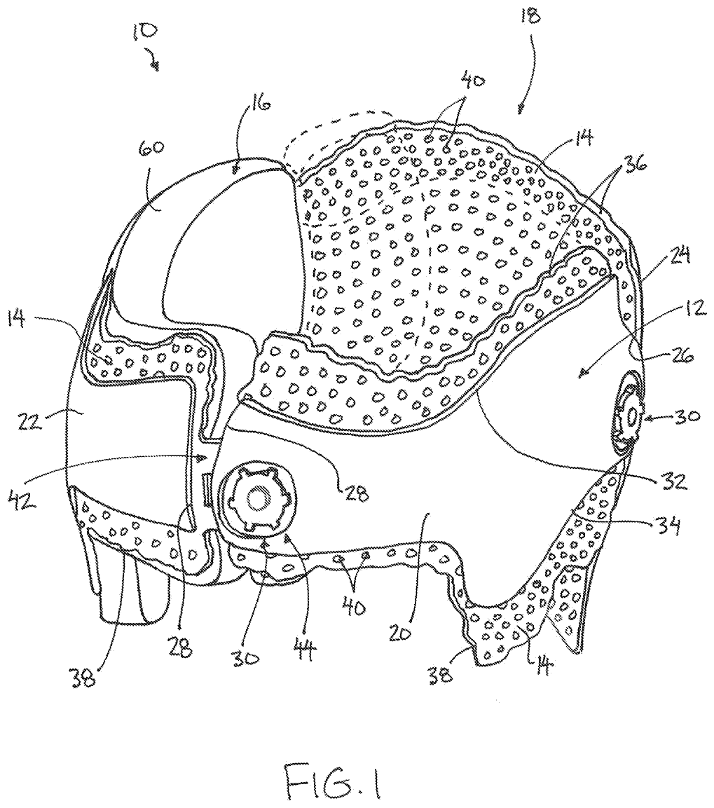

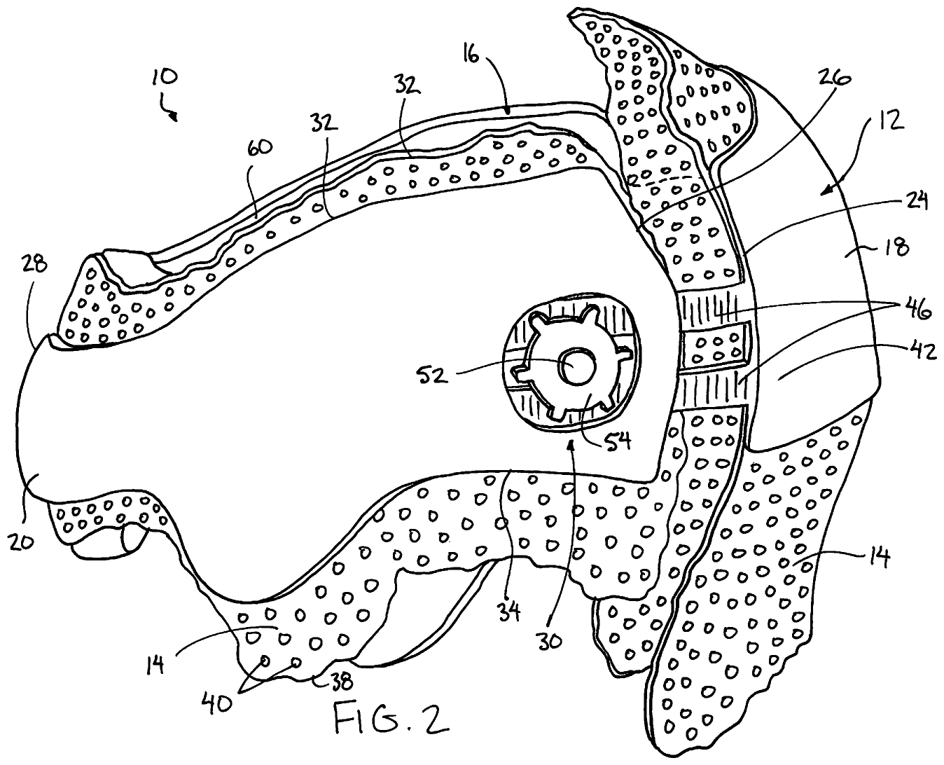

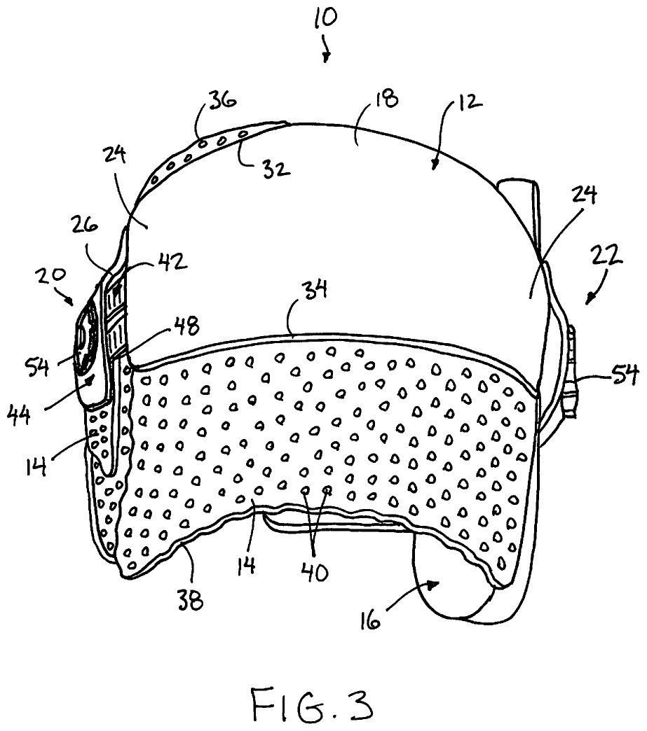

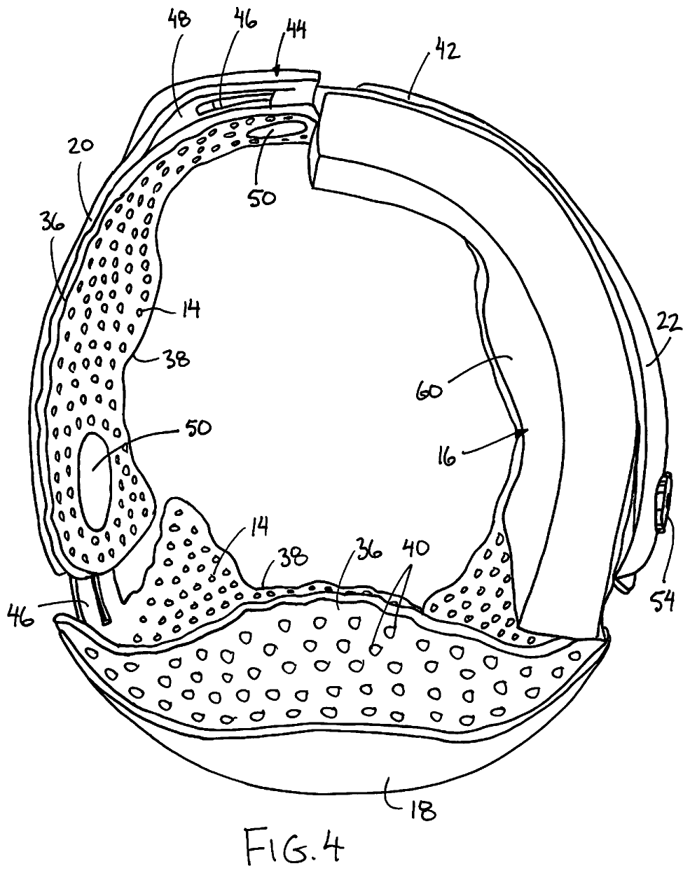

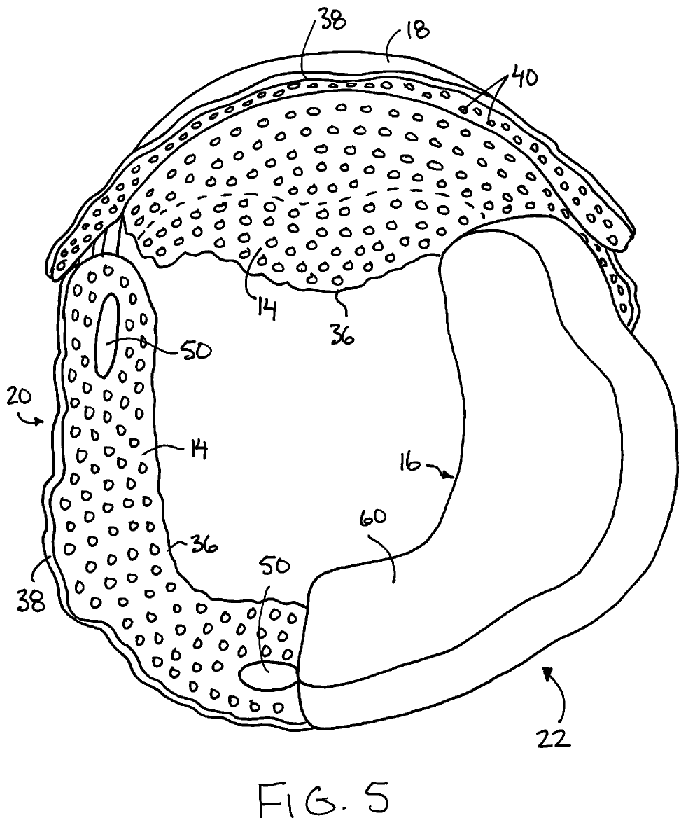

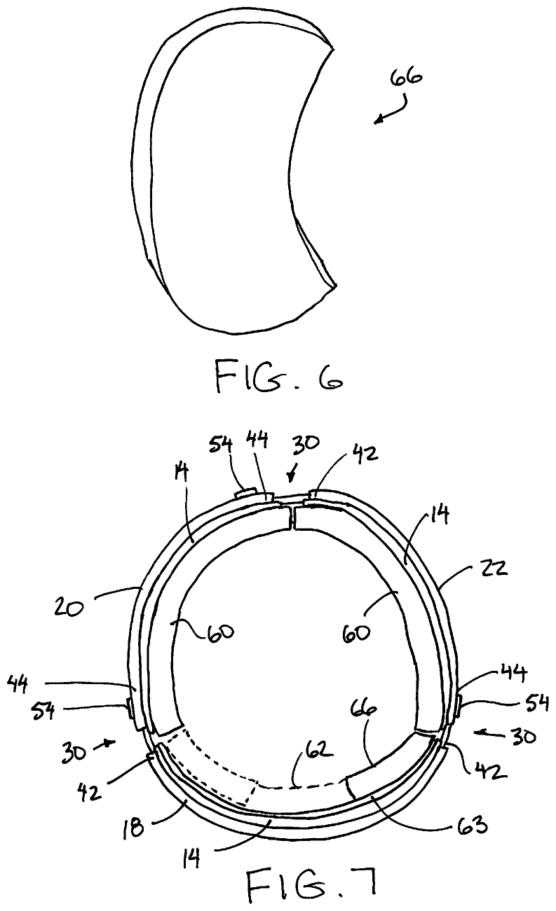

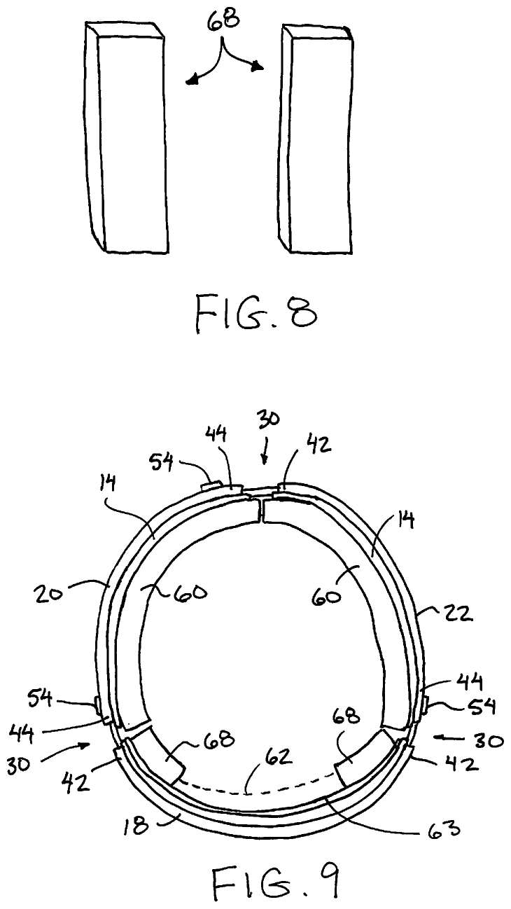

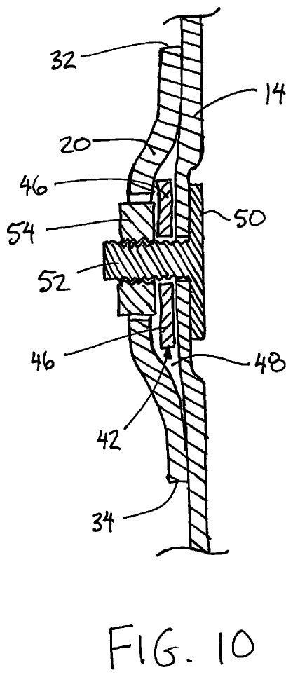

In the drawings like characters of reference indicate corresponding parts in the different figures.

### DETAILED DESCRIPTION

Referring to the accompanying figures there is illustrated a cranial remodelling orthotic apparatus generally indicated by reference numeral 10. The apparatus 10 is suited for mass manufacturing and subsequent customization to the patient for orthotic cranioplasty treatment to passively correct plagiocephaly and brachycephaly conditions in infants.

The apparatus 10 typically includes (i) a main frame assembly 12 comprising a rigid material that is assembled to form an annular band having tensile strength in a circumferential direction about a cranium of the patient, (ii) one or more support members 14 carried on the main frame assembly and having respective concave inner surfaces so as to define a supportive surface at an interior of the apparatus providing support to the cranium at prescribed locations, in which the support members can be shaped upon the application of heat more readily than the main frame assembly, and (iii) a cushion layer 16 comprising a plurality of pads formed of resilient material which line the supportive surface of the one or more support members for forming contact with the cranium of the patient.

The main frame assembly 12 according to the illustrated embodiment is formed of three arcuate frame sections that are connected in series with one another about a circumference of the cranium so as to occupy respective portions of the annular band. The frame sections of the main frame assembly include a rear section 18, a left side section 20 and a right side section 22.

The rear section 18 is symmetrical between laterally opposing ends 24 so as to span across a rear of the cranium and occupy a rearward portion of the annular band. The rear section 18 of the main frame assembly comprises a rigid sheet of material that is devoid of any openings and which is shaped to have a concave inner side and a convex outer side with a uniform thickness that may be in the range of 1 to 4 mm and is preferably between 2 and 3 mm in thickness between the inner and outer sides thereof.

The side sections 20 and 22 are symmetrical relative to one another such that each side section is substantially a mirror image of the other. Each side section is joined at a rear edge 26 to a respective one of the opposing ends 24 of the rear section 18. Side sections each extend forwardly from the rear edges thereof to respective front edges 28 which are adjacent to one another at a central location corresponding to the forehead of the patient. The front ends 28 are joined together such that all three arcuate frame sections are connected in series with one another in the circumferential direction.

An adjustable connector 30 is connected at each junction between an adjacent pair of the frame sections of the main frame assembly. Accordingly, the rear of each side section 20 or 22 is connected to the respective end of the rear section 18 by a respective one of the adjustable connectors 30, and the front ends 28 are similarly coupled to one another by one of the adjustable connectors 30. Each adjustable connector 30 is operable between an open position limiting relative movement between the arcuate sections of the respective adjacent pair to a linear sliding movement in the circumferential direction, and a closed position securing the arcuate sections at the respective junction in fixed relation to one another at a selected spacing in the circumferential direction between the arcuate frame sections. The configuration of the adjustable connectors 30 is described in further detail below.

The resulting annular band formed by connecting the arcuate frame sections to one another results in the frame assembly being adjustable in length between opposing front and back ends of the apparatus primarily by adjustment of the adjustable connectors 30 at opposing sides of the rear frame section, while also being adjustable in width perpendicularly to the length primarily by using the adjustable connector 30 between the side sections at the front of the apparatus. Any combination of adjustments may be performed by the adjustable connectors 30 to adjust the overall circumference while maintaining symmetry between laterally opposing sides of the apparatus 10 due to the placement of the junctions of the frame assembly.

More particularly the junctions between adjacent frame sections of the main frame assembly are situated such that a forward junction is located at the front end of the annular band at a laterally central location and such that the side junctions at laterally opposing sides of the apparatus in proximity to the rear end thereof are situated rearward of the ear area of the patient in a manner that does not align with the seams between adjacent bones forming the cranium of the patient.

Each of the frame sections of the main frame assembly include a top edge 32 and a bottom edge 34 extending generally in the circumferential direction such that the top edges of the frame sections collectively define an upper boundary of the frame assembly while the bottom edges 34 of the frame sections collectively define a lower boundary of the frame assembly about the circumference of the annular band.

An overall height of the main frame assembly between the top and bottom edges is reduced at the front end where the main frame assembly extends across the forehead as compared to the overall height of the main frame assembly between the top and bottom edges at the rear end where the main frame assembly spans over a rear portion of the cranium. The bottom edges 34 of the side sections 20 and 22 may be contoured to include a depending lobe at an intermediate location between the forehead and the respective ear of the patient such that the depending lobe is forward of the respective ear.

One of the support members 14 is provided on each arcuate frame section of the main frame assembly. Each support member 14 is mounted on the frame section so as to fully span the inner side of the frame section. The support member protrudes both above the upper boundary formed by the top edge 32 of the frame section to a respective upper free edge 36 of the support member and below the lower boundary formed by the bottom edge 34 of the frame section to a respective lower free edge 38 of the support member. The upper free edges 36 of the support members thus collectively form an upper boundary of the support members which is above the upper boundary of the main frame assembly about the full circumference of the apparatus. Similarly, the lower free edges 38 of the support members collectively form a lower boundary of the support members which is below the lower boundary of the main frame assembly about the full circumference of the apparatus. The support members may also protrude in the circumferential direction beyond the opposing ends of the corresponding arcuate frame section upon which it is supported.

Each support member comprises a sheet formed of a rigid material which is the same material forming the rigid sheets of the frame sections of the main frame assembly. The rigid sheet forming each support member has a uniform thickness between the inner and outer sides thereof which may be between 1 and 4 mm, and more preferably between 2 and 3 mm in thickness.

Each support member 14 is joined to the inner side of the corresponding frame section upon which it is supported such that the support member and the respective frame section form a single body. In one embodiment the support member and the respective frame section upon which it is supported are formed together integrally and seamlessly so as to be continuous with one another as a uniform body of material throughout. For example, the structure of each support member 14 and the corresponding frame section of the main frame assembly may be 3D printed together as a singular body. Alternatively, each support member 14 and the corresponding frame section upon which it is supported may be separately manufactured and then joined to one another, for example by adhesive or by the application of heat which melts or welds the two components together to form a singular body of common material which is seamless and continuous throughout. With the support member 14 being joined to the inner side of the corresponding frame section, the overall thickness of the rigid supportive material is formed as double layer where the support member overlaps the frame section but as a single layer where the support member 14 protrudes above and/or below the corresponding frame section.

Each support member 14 is perforated by a uniform array of perforations 40. Where there is a single layer of material formed solely by the support member 14 protruding above or below the corresponding frame section, the perforations extend fully through the rigid sheet forming the support member. At the region of overlap of each support member with the corresponding frame section, the non-perforated frame section that is devoid of openings effectively encloses any perforations within the corresponding overlapped region of the support member 14.

The support member is formed of a suitable plastic material that remains rigid until the application of heat which allows the support member to become malleable and readily shaped prior to once again becoming rigid once the material cools. The material of the support member 14 may become malleable when heated by a conventional hairdryer capable of generating a heated air flow of approximately 50° C. for example. Due to the combined thickness of material of the support member and the corresponding frame section at the region of overlap therebetween as compared to the single layer portions of the support member protruding beyond the frame section, together with the additional perforations in the support member as compared to the corresponding frame section, upon the application of heat by a heated air flow, the protruding portions of the support member will be heated much more quickly. The protruding portions of the support member will thus be much more readily shapable under the application of heat while the corresponding frame section and portions of the support member connected thereto remain rigid and unaffected by the application of the heat. In this manner, the main frame assembly remains substantially rigid while the contour of the support members can be changed upon the application of heat to vary the overall curvature of the concave inner surface of the support members. The overall height of the support members can also be changed by curling the upper free edges or the lower free edges thereof outwardly and inwardly towards the opposing free edge.

Each adjustable connector 30 forms a connection between a male end 42 at the end of one of the frame sections at the corresponding junction and a female end 44 at the end of the other frame section at the corresponding junction. Each male end of a frame section comprises two flanges 46 which protrude beyond the end of the frame section in the circumferential direction while being spaced apart one above the other to define a longitudinally slot therebetween. The flanges 46 can be formed integrally with one or both of the frame section or the support member 14 carried on the respective frame section.

The female end 44 of each adjustable connector 30 comprises a pocket 48 formed between the sheet forming the support member 14 and the sheet forming the corresponding frame section. More particularly, the rigid sheet forming the frame section is bulged outward from the sheet of the support member to define an open-ended pocket that slidably receives the pair of flanges 46 of the corresponding male end 42 of the connector slidable therein in the circumferential direction.

At the location of the pocket, a bolt is provided for fixing the flanges of the male end 42 within the pocket of the female end 44 at any one of a plurality of selected positions within a range corresponding to different spacings in the circumferential direction between the frame sections joined by the adjustable connector. The bolt includes a head flange 50 received at the inner side of the support member within a corresponding recess such that the head flange 50 is substantially flush with the remaining inner surface of the support member 14. A shaft 52 of the bolt extends through a corresponding aperture for alignment with a longitudinal slot formed between the two flanges 46 of the male end 42. A suitable nut 54 is recessed into an opening at the other side of the frame section for threading onto the shaft 52 at the exterior of the apparatus. Tightening the nut onto the shaft causes the adjustable clamp to be closed by effectively clamping and fixing the flanges 46 of the male end 42 in fixed relation to the support member 14 of the female end 44.

At each of the side junctions located at the rear edges of the side sections 20 and 22, a male end 42 is formed on the rear section 18 while the rear end of each side section defines a female end 44. At the front of the apparatus, a male end is provided at the front of one side section while a female end is provided at the front of the other side section.

The cushion layer 16 includes a cushion pad 60 fully spanning the support member 14 of each side section 20 and 22. The cushion pad 60 is sized to fully line the inner supportive surface of the support member and to extend beyond the perimeter edge of the support member in all directions to prevent any sharp edges of the support member from directly engaging the patient. The cushion pad is a resilient foam material having a uniform thickness throughout, for example 7/16 of an inch.

The cushion layer also includes a neck pad (not shown) which is generally rectangular in shape and which is formed of the same foam with the same thickness as the cushion pads 60. The neck pad is arranged to extend across the full width of the rear support member 14 on the rear frame section 18 between laterally opposing ends thereof but has a height which only corresponds to a bottom portion of the rear support member 14 to be positioned adjacent the lower free edge thereof.

A liner pad (not shown) is further provided of a denser and thinner resilient foam material than the cushion pad so as to have a higher durometer than the cushion pad. The liner pad fully spans the inner surface of the support member 14 on the rear frame section 18 above the neck pad to prevent any direct contact of the rigid supportive material of the support member with the patient.

The cushion layer is typically also provided with one or more control pads which are interchangeably supported at the interior of the rear frame section for treating different conditions.

The control pads include a plagiocephaly control pad 66 for treating a plagiocephaly condition. The control pad 66 is formed of the same resilient foam material having a same thickness as the cushion pads 60. The control pad 66 is generally kidney shaped having a concave inner edge which mates in close abutment with a corresponding convex rear edge of one of the cushion pads 60. As shown in solid line in FIG. 7, the control pad 66 may be located at the right side of the support member on the rear section 18 while maintaining a circumferential gap between the control pad 66 and the cushion pad 60 at the laterally opposing side of the apparatus. Alternatively as shown in broken line in FIG. 7, the control pad 66 may be located at the left side of the support member on the rear section 18 while maintaining a circumferential gap between the control pad 66 and the cushion pad 60 at the laterally opposing side of the apparatus.

The control pads also include two brachycephaly control pads 68 for treating a brachycephaly condition. The control pads 68 are formed of the same resilient foam material having a same thickness as the cushion pads 60. The control pads 68 are each rectangular in shape so as to be elongate between opposing top and bottom ends. In use, the control pads 68 span a height of the support member 14 on the rear section 18 at laterally opposing sides thereof such that each control pad 68 abuts with the rear edge of a corresponding one of the cushion pads 60 on the side sections of the apparatus. A gap remains in the circumferential direction between the two control pads 68.

The apparatus 10 according to the present invention can be manufactured in mass quantities at a standardized shape and size for accommodating all patients. Subsequent to manufacturing, the apparatus 10 can be fitted to a particular patient by simple adjustment of the adjustable connectors 30 and minor reshaping of the protruding portions of the support members 14 using application of heat that allows the support members to be more readily shaped than the frame sections of the main frame assembly. The cushion members can be readily trimmed to size according to the resizing of the frame assembly. The appropriate control pads 66 or 68 are selected and can also be trimmed to further customize the contact areas of the apparatus with the cranium of the patient. Treatment occurs in a passive manner by allowing cranium growth into the voided areas that are devoid of any thick cushioning. The same apparatus 10 can be used for an extended period of time by gradually increasing the overall size in length and width in increments corresponding to the growth of the cranium of the patient.

To accommodate a wide variety of head shapes, the apparatus 10 is fully adjustable in both width and length in a manner unseen in previous devices. The use of a nut forming an external dial for each connector together with a slider mechanism, allows the orthotic to be adjustable in infinite increments between 400 and 500 mm of circumference while maintaining physical integrity and strength. Three uniquely shaped and strategically placed seams ensure comfortable fitment of the orthotic throughout the adjustment range while ensuring optimal contact points are used to improve skull symmetry and obtain the desired combination of width and length adjustability required. Each of the three sections of the apparatus provides a layering of (i) a soft inner foam shell to adapt to the more minute, unique shapes of an individual skull, (ii) a perforated heat mouldable layer formed by the support member 14 that is designed to increase surface area of contact between the orthotic and the skull and provide individual height and shape adjustability that allows for treatment of both plagiocephaly and brachycephaly head shapes, and (iii) a rigid external frame that provide structural integrity to the orthotic and houses the adjustment mechanisms. The apparatus 10 also includes specially designed cranial contact control pads that allow for customization based on the type of skull asymmetry presented.

Since various modifications can be made in my invention as herein above described, and many apparently widely different embodiments of same made, it is intended that all matter contained in the accompanying specification shall be interpreted as illustrative only and not in a limiting sense.

1. A cranial remodeling orthotic for passive remodeling of a cranium of an infant, the orthotic comprising:
a main frame assembly forming an annular band having an upper boundary and a lower boundary so as to be arranged to encircle the cranium of the infant;
at least one support member supported on the main frame assembly and having a concave inner surface, said at least one support member being arranged to extend circumferentially with the main frame assembly about a respective portion of the cranium of the infant, and said at least one support member protruding beyond at least one of the upper boundary or the lower boundary of the main frame assembly;
said at least one support member being formed of a support material that is rigid and self supporting; and
a cushion layer formed of resilient material lining an inner surface of at least a portion of the main frame assembly or said at least one support member;
the support material of said at least one support member being formable in shape upon the application of heat; and
said at least one support member being sized and configured so as to be formable in shape upon the application of heat more readily than the main frame assembly.

2. The orthotic according to claim 1 wherein said at least one support member protrudes above the upper boundary of the main frame assembly.

3. The orthotic according to claim 2 wherein said at least one support member protrudes above the upper boundary of the main frame assembly about a full circumference of the main frame assembly.

4. The orthotic according to claim 1 wherein said at least one support member protrudes below the lower boundary of the main frame assembly.

5. The orthotic according to claim 4 wherein said at least one support member protrudes below the lower boundary of the main frame assembly about a full circumference of the main frame assembly.

6. The orthotic according to claim 1 wherein said at least one support member is perforated by an array of openings.

7. The orthotic according to claim 1 wherein the main frame assembly and said at least one support member are both formed of said support material, and wherein the main frame assembly has fewer openings therein than said at least one support member such that said at least one support member is formable in shape upon the application of heat more readily than the main frame assembly.

8. The orthotic according to claim 1 wherein said at least one support member has at least one of an upper free edge above the main frame assembly or a lower free edge below the main frame assembly that can be curled outward relative to the main frame assembly upon the application of heat.

9. The orthotic according to claim 1 wherein said at least one support member fully spans an inner side of the main frame assembly.

10. The orthotic according to claim 1 wherein said at least one support member is formed integrally and seamlessly of the same material with a respective portion of the main frame assembly.

11. The orthotic according to claim 1 wherein the main frame assembly comprises a plurality of arcuate sections connected circumferentially in series with one another to form the annular band, said at least one support member comprising one or more support members supported on each arcuate section of the main frame.

12. The orthotic according to claim 1 wherein the main frame assembly comprises (i) a plurality of arcuate sections connected circumferentially in series with one another at a plurality of junctions to form the annular band and (ii) an adjustable connector at each junction between a respective adjacent pair of the arcuate sections, each adjustable connector being adjustable in length in a circumferential direction of the annular band and being securable to join the adjacent pair of arcuate sections in fixed relation to one another, the arcuate sections enabling the annular band to be adjusted in length between a front and a rear of the annular band and to be adjusted in width transversely to the length.

13. The orthotic according to claim 12 wherein each adjustable connector is operable between an open position limiting relative movement between the arcuate sections of the respective adjacent pair to linear sliding movement in the circumferential direction and a closed position securing the arcuate sections of the respective adjacent pair in fixed relation to one another.

14. The orthotic according to claim 1 wherein the main frame assembly comprises a plurality of arcuate sections connected circumferentially in series with one another to form the annular band, the plurality of arcuate sections including (i) a rear section occupying a rear of the annular band for alignment with a rear of the cranium of the infant and (ii) two side sections extending forwardly from opposing ends of the rear section respectively so as to occupy opposing side portions of the annular band respectively, the two side sections being joined to one another at a front of the annular band for alignment with a forehead of the cranium of the infant.

15. The orthotic according to claim 14 wherein the cushion layer comprises a cushion pad supported on each of the two side sections and at least one control pad releasably supported on the rear section.

16. The orthotic according to claim 15 wherein the at least one control pad comprises:
(i) a plagiocephaly control pad arranged to be supported along only one side of the rear section of the annular band, the plagiocephaly control pad being releasably supported on the rear section so as to allow placement on the rear section adjacent to either one of the two side sections; or
(ii) two brachycephaly control pads arranged to be supported at circumferentially spaced positions on the rear section of the annular band, adjacent to the two side sections of the annular band respectively.

17. A cranial remodeling orthotic for passive remodeling of a cranium of an infant, the orthotic comprising:
a main frame assembly forming an annular band arranged to encircle the cranium of the infant;
at least one support member supported on the main frame assembly and having a concave inner surface, said at least one support member being arranged to extend circumferentially with the main frame assembly about a respective portion of the cranium of the infant, said at least one support member being formed of a support material that is rigid and self supporting; and
a cushion layer formed of resilient material lining an inner surface of at least a portion of the main frame assembly or said at least one support member;
the main frame assembly comprises a plurality of arcuate sections connected circumferentially in series with one another to form the annular band, the plurality of arcuate sections including:
(i) a rear section occupying a rear of the annular band for alignment with a rear of the cranium of the infant; and
(ii) two side sections extending forwardly from opposing ends of the rear section respectively so as to occupy opposing side portions of the annular band respectively;
the two side sections being joined to one another at a front of the annular band for alignment with a forehead of the cranium of the infant.

18. The orthotic according to claim 17 wherein the cushion layer comprises a cushion pad supported on each of the two side sections and at least one control pad releasably supported on the rear section.

19. The orthotic according to claim 18 wherein the at least one control pad comprises a plagiocephaly control pad arranged to be supported along only one side of the rear section of the annular band, the plagiocephaly control pad being releasably supported on the rear section so as to allow placement on the rear section adjacent to either one of the two side sections.

20. The orthotic according to claim 19 wherein the cushion pads and the plagiocephaly control pad are formed of a common foam material and have a common thickness in a radial direction of the annular band, the plagiocephaly control pad being circumferentially abutted against one of the cushion pads.

21. The orthotic according to claim 18 wherein the at least one control pad comprises two brachycephaly control pads arranged to be supported at circumferentially spaced positions on the rear section of the annular band, adjacent to the two side sections of the annular band respectively.

22. The orthotic according to claim 21 wherein the cushion pads and two brachycephaly control pads are formed of a common foam material and have a common thickness in a radial direction of the annular band, two brachycephaly control pads being circumferentially abutted against the two cushion pads respectively.

23. The orthotic according to claim 18 wherein the at least one control pad comprises a scaphocephaly control pad arranged to be supported directly in the mid-line of the rear section of the annular band, in which the scaphocephaly control pad is releasably supported on the rear section so as to allow placement on the rear section adjacent to either one of the two side sections.

24. The orthotic according to claim 23 wherein the cushion pads and the scaphocephaly control pad are formed of a common foam material and have a common thickness in a radial direction of the annular band, and the scaphocephaly control pad is placed evenly spaced between the two side cushion pads.
